(19) United States
(12) Patent Application Publication
(10) Pub. No.: US 2007/0244469 A1
(43) Pub. Date: Oct. 18, 2007

(54) MINIATURE INFUSION PUMP FOR A CONTROLLED DELIVERY OF MEDICATION

(76) Inventors: Shaul Ozeri, Ramat Aviv (IL); Jehonatan Ozeri, Ramat Aviv (IL)

Correspondence Address: FLEIT KAIN GIBBONS GUTMAN BONGINI & BIANCO, 21355 EAST DIXIE HIGHWAY, SUITE 115, MIAMI, FL, 33180, US

(21) Appl. No.: 11/746,658

(22) Filed: May 10, 2007

Related U.S. Application Data
(63) Continuation of application No. PCT/IL05/01185, filed on Nov. 10, 2005.
(60) Provisional application No. 60/626,948, filed on Nov. 12, 2004.

(51) Int. Cl. A61M 1/00; A61K 9/22

(52) U.S. Cl. 604/891.1; 604/152

(57) ABSTRACT

The present invention is a miniature medication pump. With a small lightweight mechanism, operating on low energy consumption, the medication pump device is a highly reliable controlled portable device of drug infusion to patients. The proposed device is comprised of a piezoelectric actuator, operated by a programmable logic means, which applies force on a lever. The lever is situated between two unidirectional stoppers and transfers the force of the actuator onto the front stopper. The front stopper has a grip on the plunger stem and it therefore pushes it in the direction of the syringe and releases controlled amounts of medication to the body of the patient. The invention also include monitoring mechanisms which enable constant and accurate measurements of the amount of medication which is released from the device in actuality. The user of the pump is alerted about detected deviations from the designated flow of the medication.

### BACKGROUND OF THE INVENTION

The present invention relates to the field of infusion pumps for controlled delivery of medication to patients, and more specifically it relates to minute infusion pumps for controlled delivery of medication to patients with an improved lightweight drive mechanism.

Infusion pumps deliver volumetrically controlled flow of medication to patients over a given period of time. A processing circuitry controls the periodic delivery of dosages of medication to a patient at predetermined rates. Syringe infusion pumps often contain an electrical motor which rotates a lead-screw; the rotation of the lead-screw causes a nut to linearly move along it. The nut pushes a plunger through a syringe or a cartridge internal to the pump that causes medication to move from the syringe to the patient along the infusion path.

Prior art of Infusion pumps contain a large electrical motor which is strong enough to rotate the lead-screw against the opposing pressure of the medication inside the syringe. Such mechanism is described, for example, in U.S. Patent Application No. 20,030,205,587, and U.S. Pat. Nos. 6,248,093, 5,637,095, 5,097,122, and 5,505,709. These devices contain electrical motors which are relatively large and heavy. Since dosages are given at discrete intervals over a period of time, each time the processing circuit activates the motor it consumes a relatively high amount of energy.

In addition to the disadvantages in size, weight and power consumption of existing medication pumps, these devices also suffer from a drawback which stems from the principles according to which they operate. The amount of medication delivered from the device into the patient's body is controlled by the operation of the motor. The accuracy of these devices is therefore difficult to control and dependent on the reliability and accuracy of the motor; minute fluctuations in the motor's behavior might cause significant deviations in the amount of medication delivered to the patient. The medication delivery is therefore calculated statistically.

Elaborate devices were developed to detect and respond to inconsistent flow rates as solutions to this problem. Whenever a pressure build-up is detected inside the syringe, these devices most commonly compensate for the reduction of flow by changing the time intervals between successive pulses. If the pressure reaches an occlusion level, the pump stops and the user is alerted. This is not a satisfactory solution. Furthermore, once the blockage is opened, the pressure which is built inside the container and delivery tube is released through the tube, forcing a possibly dangerously larger than prescribed dose of medicine into the patient's body.

French Patent No. 2728172 discloses an injection cartridge comprising a barrel containing the substances to be injected, situated between a forward wall equipped with a needle, and a rear wall which acts as the piston. The forward wall forms part of a mobile assembly which moves inside the barrel by means of an actuator between a retracted position inside the barrel and an active position in which the needle projects beyond the barrel.

International Patent Application No. 03103763 discloses a device for delivering medication to a patient. The device includes an exit port assembly, a syringe-like reservoir including a side wall extending along a longitudinal axis towards an outlet connected to the exit port assembly, and a plunger assembly received in the reservoir. The plunger assembly includes a longitudinal segment connecting the first and second lateral segments. The longitudinal segment includes a spring biasing the first and the second lateral segments apart, and an actuator arranged to overcome the spring and bias the first and second lateral segments longitudinally together upon actuation. Successively actuating the actuator causes longitudinal movement of the plunger assembly together towards the outlet of the reservoir in order to cause fluid to be dispensed from the reservoir to the exit port assembly.

According to U.S. Patent Application No. 2004124214 in a cartridge for a fluid, as well as a system for handling a fluid using such a cartridge, the cartridge has a tank shaped as a cylinder with an opening to admit and discharge the fluid, as well as a piston that can be moved forward and/or back in the tank in order to pump the fluid in or out through the opening. The piston has a connection element that can be connected with an actuator in order to move the piston forward and/or back. The connection element and the actuator are adapted to one another such that the connection is automatically closed in a first longitudinal section of the tank, given movement of the piston in the longitudinal direction and is automatically released again given movement in the opposite direction, and the connection remains closed in a second longitudinal section of the tank given movement of the piston into this second longitudinal section.

There is therefore a need for a medication pump, which, in addition to being very small, lightweight and low in energy consumption, is able to deliver accurate and consistent dosage rate of medication over periods of time. Such medication pumps should also include safety features which accurately monitor the pressure applied by the pump and the flow of medication it generates.

### SUMMARY OF THE INVENTION

The present invention discloses a micro pump device for dispensing proportioned quantities of medical fluid by applying pulsed pressure on the plunger stem of a syringe containing medical fluid which is injected through a syringe-tube connector to the body of the patient. The device is comprised of a levering mechanism for applying pressure on the plunger stem in the direction of the tube of the syringe. The lever mechanism includes a lever revolving around a fixed axis and two unidirectional stoppers, a front stopper and a rear stopper. The movement of the lever applies pressure on said front stopper.

The device is also comprised of an expending actuator means for applying pressure on the levering mechanism. The activation of the actuator is controlled by a programmable logic module. The first end of the lever is in contact with the actuator and the second end is in contact with the front unidirectional stopper.

The unidirectional stoppers comprise a loop placed around the plunger stem having an inner diameter slightly larger then the outer diameter of the plunger stem, and a spring. The spring of front unidirectional stopper returns said loop to its initial position in relation to said lever. The level is located in between the rear stopper and the front stopper. The rear unidirectional stopper prevents the backwards motion of the plunger.

The device also includes a knob for releasing the hold of the second unidirectional stopper on the plunger stem by slightly changing the angle of the loop of the rear stopper and releasing its grip on the plunger stem. Moving the knob enables the refill of the syringe tube or the replace of it. Upon releasing the knob the spring of the rear stopper returns the loop of the rear stopper back into place in relation to the plunger stem.

The micro pump device also includes a sensor for measuring the force of the actuator. The sensor may be positioned between the actuator and a stopper which is holding one end of the actuator in place. Alternatively, the sensor may be positioned between the plunger stem and the seal of the syringe. The sensor may be a piezoelectric transducer.

The micro pump device may also include a sensor for monitoring the movement of the plunger stem, enabling a continuous calculation of the pump flow rate. This sensor may be an optical sensor or an acoustic sensor. The acoustic sensor is comprised of a piezoelectric transducer, acoustical wave guide, acoustical mirror attached to the plunger and two matching layers. The sensor measures the position of the plunger stem at any given moment in accordance with the time interval of a signal traveling between the piezoelectric transducer and the mirror. The acoustic sensor may also include a temperature sensor for calibrating the acoustical measurement. A mechanical obstacle may be positioned in the wave guide at a fixed position providing a reference measurement for correcting the acoustical measurement. Alternatively, the sensor may be a capacitive sensor or an electromagnetic sensor positioned around the rear end of the plunger stem. The electromagnetic sensor is a linear resolver comprised of a windings and a core.

The programmable logic module of the device includes a current fed single stage actuator charging circuit. The programmable logic module may include a single stage boost step-up actuator charging circuit, a charging circuit using a boost step-up and a piezoelectric transformer or an actuator dissipative discharging circuit.

The device may be connected to a transdermal disposable passive patch, an active transdermal sonophoretic patch or an active transdermal multi needle array patch. The multi array patch may mechanically vibrate to improve the medication delivery of the patch. The multi array patch may contain a piezoelectric vibrating element or electromagnetic vibrating element. The delivery patches can communicate with the pump alerting in cases of unexpected events and increasing the safety of the device. The controller may also communicate with a glucose sensor to form a closed loop system. The spring may be made of plastic, rubber or a metal material.

The device may be carried by the user using a pouch. The energy source of the device may be a self generating electrical energy source by mechanical means or by using a piezoelectric element which converts mechanical vibrations to electrical energy. The device may further include a cellular communication module for control and indications and a GPS module which can locate the patient. The device may also include a soft sealing around the plunger stem that prevents contamination from entering the mechanism from said syringe tube.

### BRIEF DESCRIPTION OF THE DRAWINGS

These and further features and advantages of the invention will become more clearly understood in the light of the ensuing description of a preferred embodiment thereof, given by way of example, with reference to the accompanying drawings, wherein—

FIG. 1 is an illustration of the principle components of the device according to the preferred embodiment of the present invention;

FIG. 2 is a detailed illustration of the syringe plunger pushing mechanism according to the preferred embodiment of the present invention;

FIG. 3 is a detailed illustration of the rear-end monitoring mechanism according to the preferred embodiment of the present invention;

FIG. 4 is an illustration of the magnetic resolver circuit according to the preferred embodiment of the present invention;

FIG. 5 is an illustration of the waveforms of the magnetic resolver according to an embodiment of the present invention;

FIG. 6 is an illustration of a single stage boost step-up actuator charging circuit according to an embodiment of the present invention;

FIG. 7 is an illustration of a current fed single stage actuator charging circuit according to an embodiment of the present invention;

FIG. 8 is an illustration of a two boost converters in series actuator charging circuit which reduce duty cycle range according to an embodiment of the present invention;

FIG. 9 is an illustration of an actuator charging circuit using a boost step-up and a piezoelectric transformer according to an embodiment of the present invention;

FIG. 10 is an illustration of an actuator dissipative discharging circuit according to an embodiment of the present invention;

FIG. 11 is an illustration of an actuator non-dissipative resonant discharging circuit according to an embodiment of the present invention;

FIG. 12 is an illustration of the acoustic resolver according to an embodiment of the present invention;

FIG. 13 is a detailed illustration of the components of the acoustic resolver according to an embodiment of the present invention;

FIG. 14 is a detailed illustration of the components of the acoustic resolver including a fixed obstacle for creating a reference signal according to an embodiment of the present invention;

FIG. 15 is an illustration of the location of the transducer of the monitoring mechanism according to a preferred embodiment of the present invention.

FIG. 16 is a diagram illustrating the circuitry of a sensor which is able to measure static mechanical force or a force whose magnitude is changing very slowly, which is within the scope of the present invention;

FIG. 17 is a diagram of a circuitry of the RLC electric equivalent of a piezoelectric element which is designed to measure static mechanical force;

FIG. 18 is an illustration of a charge-discharge diagram of the static piezoelectric element;

FIG. 19 is an illustration of the zero voltage which is forced on the electrical electrodes in the static piezoelectric element;

FIG. 20a and FIG. 20b illustrate possible vibration directions of the static piezoelectric element;

FIG. 21 illustrates an additional embodiment of the the syringe plunger pushing mechanism according the present invention;

### DETAILED DESCRIPTION OF THE PREFERRED EMBODIMENTS

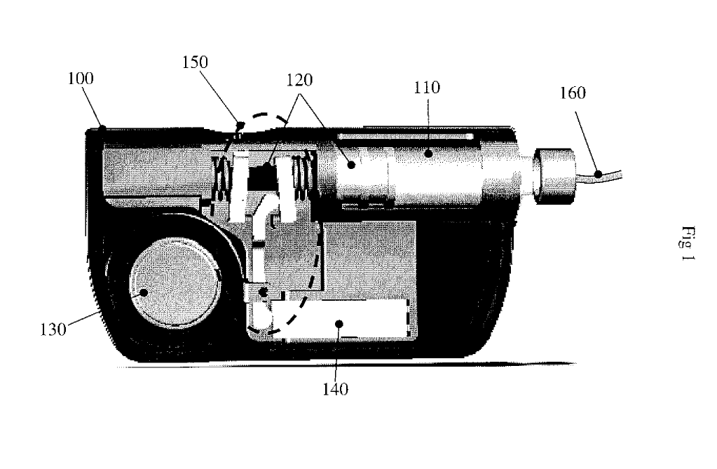

The present invention is a new and innovative miniature medication pump. With a small lightweight mechanism, which operates on low energy consumption, the medication pump device is a highly reliable controlled portable device of drug infusion to patients. The proposed mechanism does not restrict the motion and mobility of its users and allows patients using it to easily conceal the device. The pumping device is illustrated in FIG. 1. Device 100 is comprised of syringe 110 and its plunger 120. The medication fluid flows from syringe 110 to the patient's body (not shown) through flexible tube 160 as a result of pressure which is applied on plunger 120 by mechanism 150. Operated by actuator 140, which is controlled by programmable logic means and receives energy from local energy resource 130, mechanism 150 applies pulses of force on plunger 120 in order to release a single drop of medication ranging between several nano-liters to several micro-liters of medication from the syringe to the patient. Actuator 140 may be any kind of material with piezoelectrical or ferroelectrical properties, such as a piezoelectric PZT actuator; electromagnetic and shape memory alloy actuators may also be adequate.

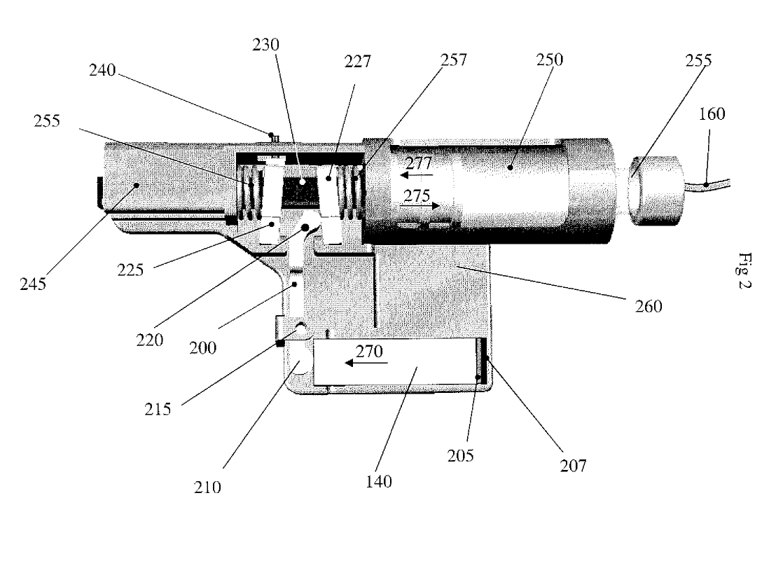

A more detailed illustration of the operation of mechanism 150 is illustrated in FIG. 2. Since it is held in place at its other end, upon receiving an electrical signal actuator 140 expands in the direction of arrow 270 towards mechanism 150. This expansion applies force on the tip of the short end 210 of lever 200, which revolves around axis 215, and therefore creates a clockwise movement in lever 200, pushing tip 220 of its long end in the direction of arrow 275. This movement of tip 220 applies force on front loop 227. Front loop 227 and rear loop 225 operate as unidirectional stoppers in opposite directions. The interior diameter of loops 225, 227 is slightly larger then that of plunger stem 230, and the angle of loops 225, 227 in relation to plunger stem 230 determine the direction of the stopper. Front loop 227 has a grip on plunger stem 230 when movement is in the direction of arrow 275, but it allows plunger stem 230 to freely move in the direction of arrow 277. Similarly, rear loop enables uninterrupted movement of plunger stem in the direction of arrow 275, but does not allow the stem to move in the direction of arrow 277. Thus, as tip 220 pushes front loop 227, loop 227 has a grip on plunger stem 230 and it pushes plunger stem 230 in the direction of arrow 275. Once the force applied by tip 220 on front loop 227 stops, spring 257 pushes front loop 227 back into its initial position.

To summarize this sequence of movements it can be said that the expansion of actuator 140 causes lever 200 to turn clockwise, and tip 220 of the long end of lever 200 pushes loop 227 in the direction of arrow 275. Since loop 227 has a grip on plunger stem 230, the force of lever tip 220 on loop 227 is transferred to plunger stem 230. Plunger stem 230 is then pushed in the direction of arrow 275 towards tube 250 of the syringe forcing the medication fluid in it out through flexible tube 160 to the patient's body. After reaching its appropriate length actuator 140 contracts and lever 200 turns back counterclockwise. Front spring 257 pushes front loop 227 to its initial position. The pressure of the medication fluid in syringe's tube 250 then might push the plunger back, but the unidirectional stopping mechanism of rear loop 225 prevents this motion. The rear loop 225 allows plunger stem 230 to move only in the direction of arrow 275, towards the syringe and prevents it from moving back in the direction of arrow 277.

The span of actuator 140 may be controlled in the range of 10 um to 50 um, for example, in accordance with its drive voltage. As mentioned above, it results in the release of a single drop of medication of any volume between a nano-liter to several micro-liters, according to its calibration.

Knob 240 is used to release the hold of rear loop 225 on plunger stem 230. When the user wishes to refill the syringe with fresh medication fluid knob 240 is released and rear spring 255 causes rear loop 225 to slightly change its angle in relation to plunger stem 230. In this angle the rear loop loosely fits around plunger stem 230 and allows it to move freely. The plunger stem may then be pulled back to its initial position. Refilling may be done in one of several ways: the tube may be refilled in the standard manner in which syringes are filled—as the stem is pulled back; the syringe may be dispensable and replaced on refill with a new syringe without a plunger stem; or pre-filled replaceable capsules may be placed inside the syringe's tube.

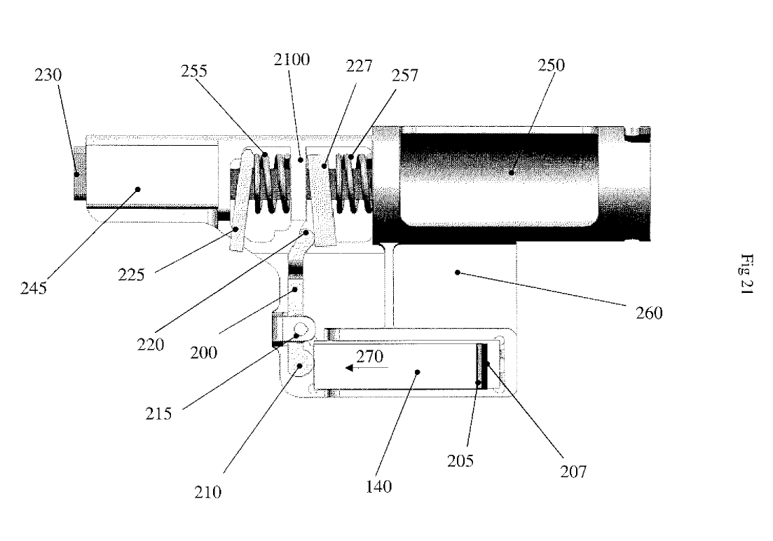

FIG. 21 illustrates an additional embodiment of the syringe plunger pushing mechanism according to the present invention. In this embodiment an additional supporting wall 2100 is positioned between rear loop 225 and front loop 227. Supporting wall 2100 enables positioning rear spring 255 in front of rear loop 225, instead of behind it. The operation of this additional embodiment is otherwise identical to that of the embodiment described above.

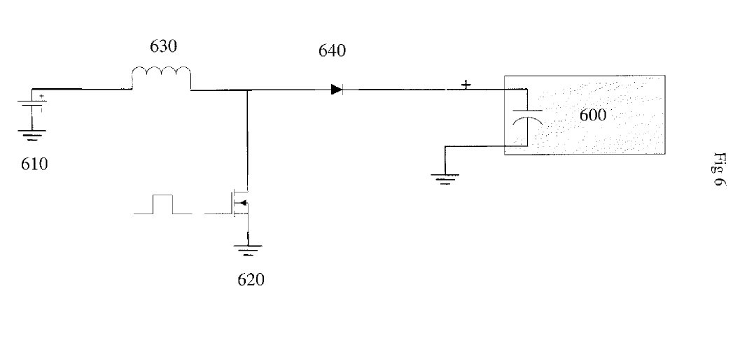

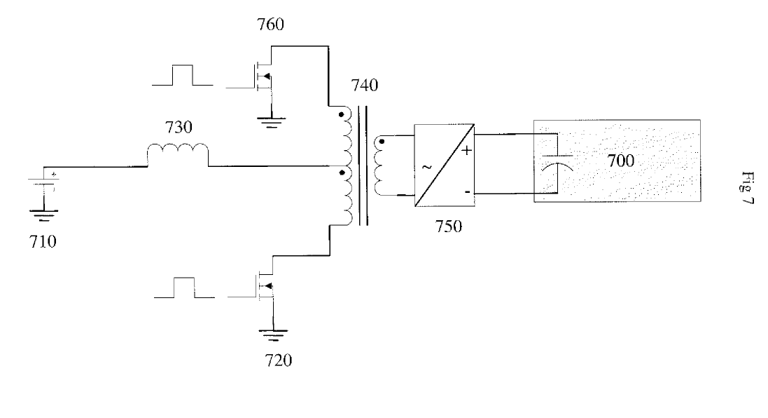

Several possible electric circuits for activating actuator 140 are illustrated in FIGS. 6 to 11. FIG. 7 is an illustration of a current fed single stage actuator charging circuit. In order to limit the duty cycle range required to increase the low battery voltage of battery 710 to approximately a 20 times higher voltage, an electrical high frequency transformer 740 is used. Inductor 730 is being charged when both transistors 720, 760 are saturated. At this phase transformer 740 is shorted by transistors 720, 760 because they force an opposing magnetic flux through transformer 740. At the second phase transistors 720, 760 are alternately blocked and the inductor 739 discharges through the transformer 740 to the actuator 700.

FIG. 6 is an illustration of a single stage boost step-up actuator charging circuit. This is a power electronics boost circuit which can step up the battery voltage of battery 610 to a higher voltage required to drive the actuator 600. Pulses at the gate of the transistor 620 cause it to conduct and the voltage at the junction common to diode 640 inductor and transistor 620 collapse to almost zero. At this time inductor 630 charges. At the second phase the control pulse of transistor 620 is removed and it is disconnected. Inductor 630 discharges partly or fully through diode 640 to actuator 600, and the voltage of actuator 600 increases. The circuit should be controlled to insure proper working parameters.

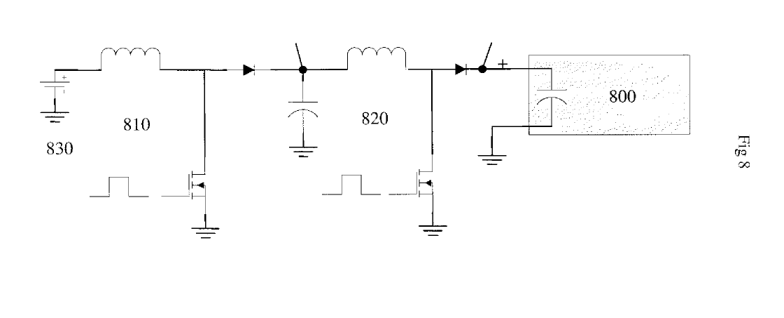

FIG. 8 is an illustration of a two boost converters in series actuator charging circuit. It is another topology which is used to limit the duty cycle range required to increase the battery voltage of battery 810. The operation of section 810 is identical to that of to the boosting circuit illustrated in FIG. 6. Section 820 is similar, but it receives as input the output voltage of section 810. Provided that section 810 increases its input voltage by the factor of A1, and section 820 increases its input voltage by the factor of A2, the total output voltage which is fed into actuator 800 is A1*A2.

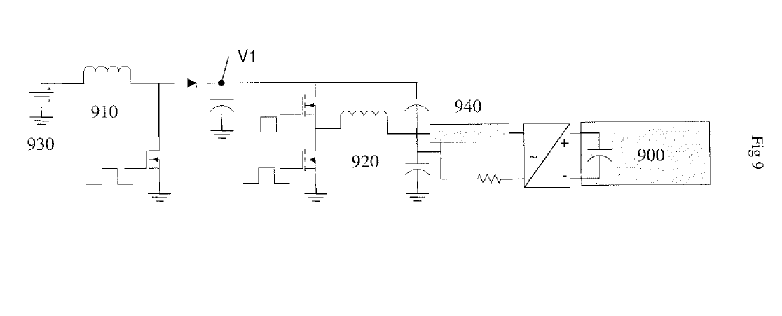

FIG. 9 is an illustration of an actuator charging circuit using a boost step-up and a piezoelectric transformer. FIG. 9 consist of a boost topology 910 which increases the battery voltage to V1. V1 is the input voltage to a second stage half bridge circuit 920 which drives piezoelectric transformer 940. The piezoelectric transformer 940 has large voltage amplification of approximately 50 times higher voltage, which is increases the initial voltage of battery 930 further. The piezoelectric transformer 940 has low profile and weight and is suitable for small portable applications.

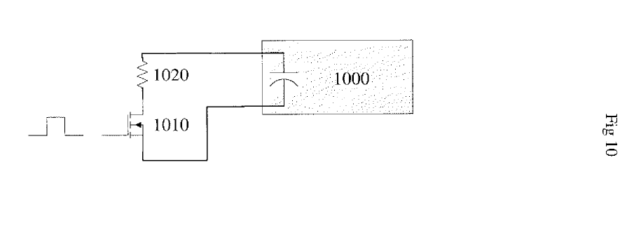

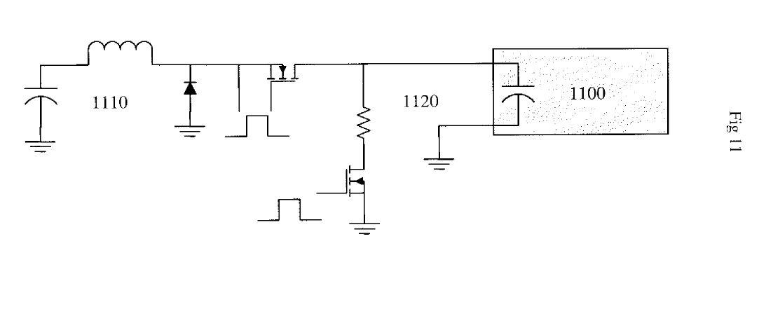

FIG. 10 is an illustration of an actuator dissipative discharging circuit. FIG. 11 is an illustration of an actuator non-dissipative resonant discharging circuit.

One critical feature of medication pumps of this sort is their ability to monitor the amounts of medication that they actually release so that the user may be warned whenever there are deviations from the preprogrammed flow rate. The preferred embodiment of the device disclosed here includes two such mechanisms.

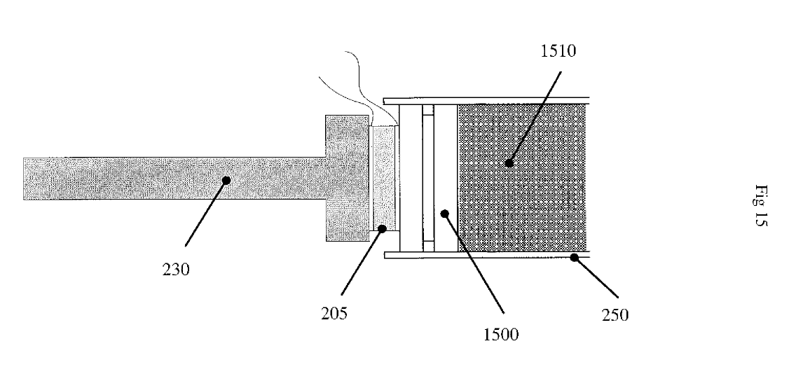

The first is a sensor which measures the amount of force which the actuator encounters as it expands. For this purpose a piezoelectric transducer may be used. A piezoelectric transducer is usually made of polycrystalline ferroelectric materials such as BaTiO3 or Lead Zirconate Titanate and translates a mechanical strain to an electric voltage at its terminals. As illustrated in FIG. 2, transducer 205 is situated at the back of actuator 140, between the actuator and actuator's stopper 207. Stopper 207 ensures that when actuator 140 expands, it only expands in the direction of arrow 270 (to the left, in this instance). Whenever actuator 140 does not behave as expected it does not produce the expected amount of force on transducer 205. Two types of malfunctions may occur: actuator 140 may produce an insufficient amount of force or produce too much force. An insufficient amount might indicate that the actuator or the electronic circuitry which operates it is not functioning properly and that the pump is not pushing a sufficient amount of medication into the patient. An unexpected increase in the amount of force indicates that the actuator is encountering too much resistance, most probably because the pressure inside syringe's flexible tube 160 is too high and some sort of blockage prevents the medication fluid from flowing into the patient. Transducer 205 may be positioned between actuator 140 and stopper 207, or as illustrated in FIG. 15, between plunger stem 230 and seal 1500. Seal 1500 is a movable part which ensures the containment of fluid 1510 inside syringe's tube 250.

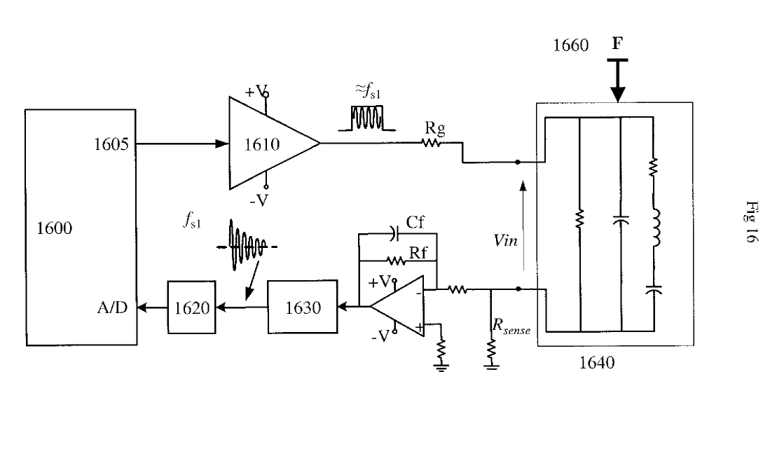

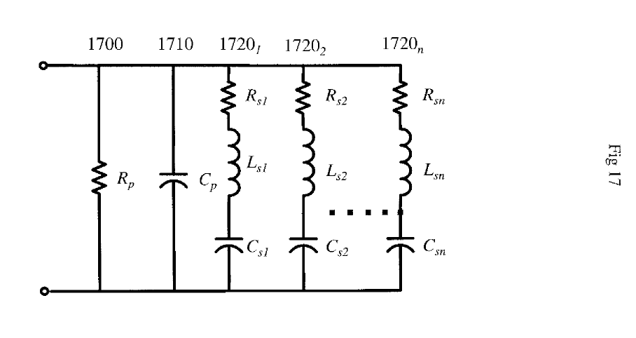

Also within the scope of the present invention is a method for performing static measurements of the pressure on a piezoelectric element, such as transducer 205. As mentioned above, the direct piezoelectric effect causes piezoelectric element 205 to generate an electrical voltage at its terminal in response to changes in the mechanical stress on it. The generated voltage amplitude is proportional to the difference in the level of the stress. This voltage decays exponentially and disappears after a while as it discharges through the equivalent parallel leakage resistance of the piezoelectric and of the electrical sensing circuitry. For this reason, normally piezoelectric elements are used to measure only dynamic mechanical force, and not static pressure. FIG. 16 is a diagram illustrating the circuitry of a sensor which is able to measure static mechanical force or a force whose magnitude is changing very slowly, like the pressure generated by the medication inside the syringe. A micro controller 1600 charges through power oscillator 1610 the piezoelectric element 1640. The static or the slowly changing force 1660 is applied on the piezoelectric element 1640. The anti-aliasing low pass filter (LPF) 1630 and the ABS complete connect the circuit back to micro controller 1600. FIG. 17 is a diagram of a circuitry of the RLC electric equivalent of a piezoelectric element 1660. The equivalent electric network contains leakage resistor 1700, dielectric capacitance 1710, and parallel RLC branches 1720₁ to 1720ₙ. Each branch 1720, which is a series resonance network with a high quality factor, corresponds to a piezoelectric mode of mechanical vibration. Branches 1720 therefore are cold motional branches. The first motional branch consists 1720₁ of and relates to the first vibration mode. When force 1660 is applied on piezoelectric element 1640, the component values of the equivalent RLC branch 1720 vary according to the level of the mechanical stress.

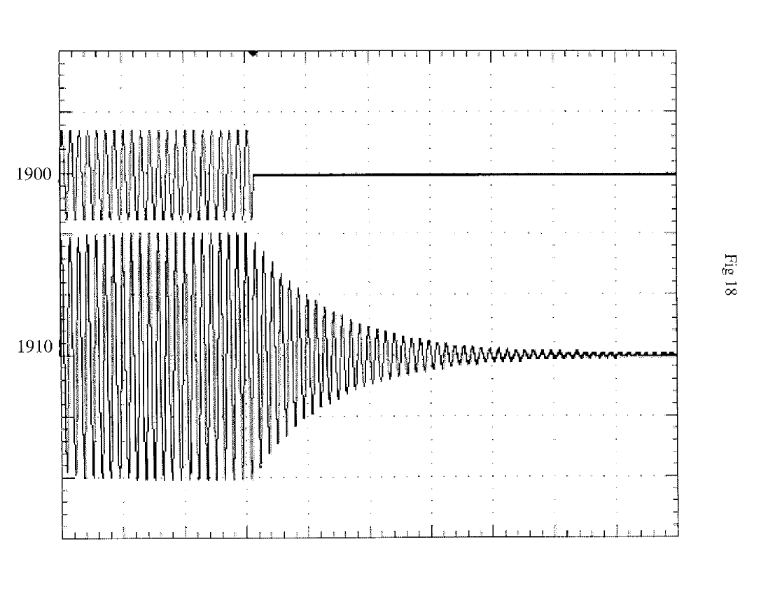

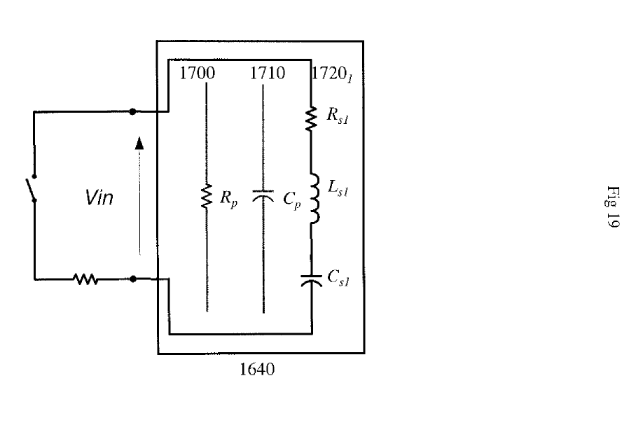

FIG. 18 is an illustration of a charge-discharge diagram of the static piezoelectric element. In order to measure the RLC value, piezoelectric 1640 is electrically stimulated with an appropriate signal which charges the RLC of the first motional branch 1720₁. After a short time-span, the stimulating signal is stopped and zero voltage is forced on the electrical electrodes, as illustrated in FIG. 19. At this point the pre-charged RLC 1720 discharges through the short circuit in an alternating current whose envelope decays exponentially. The constant level of the exponential decay depends on the level of mechanical stress 1660 exerted on element 1640. The stronger the stress 1660, the shorter decay time is. Additional indications for the magnitude of the mechanical stress can be derived from the self discharge oscillating frequency which also varies in proportion to the mechanical stress.

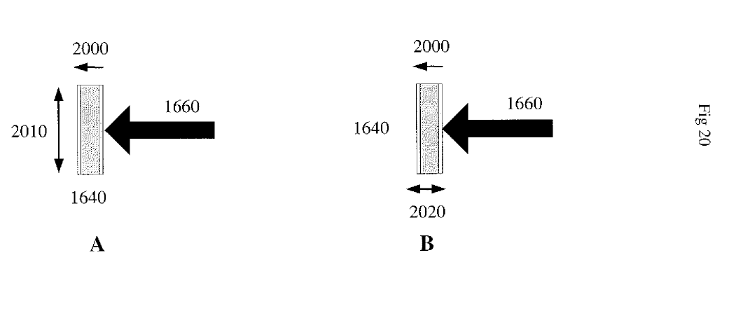

FIG. 20a and FIG. 20b illustrate possible vibration directions of the piezoelectric element. The measured mechanical force 1660 is applied on piezoelectric element 1640. The PZT polarization is in the direction of arrow 2000. As illustrated in FIG. 20a, the piezoelectric element 1640 can be forced to vibrate radially, in the direction of arrow 2010, if it is a disc shaped element. Provided that the piezoelectric element 1640 is not in the shape of a disc, it may be vibrated transversely. Additionally, as illustrated in FIG. 20b, the piezoelectric element 1640 may be vibrated longitudinally, in the direction of arrow 2020, along the axis of the applied mechanical stress 1660. The method is not restricted to measuring only the first motional branch parameters, higher vibration modes can also be used.

The second monitoring mechanism accurately locates the position of syringe stem 230 in order to monitor the progress of the stem as it is pushed in the direction of syringe's flexible tube 160 making sure that its progresses is according to plan. Moreover, it enables the controller to advance the plunger more accurately by taking into consideration data from the resolver as the pump continuously calculates the flow rates. This monitoring mechanism may be embodied in several ways—using, for instance, optic, acoustic, electromagnetic or capacitive measuring means.

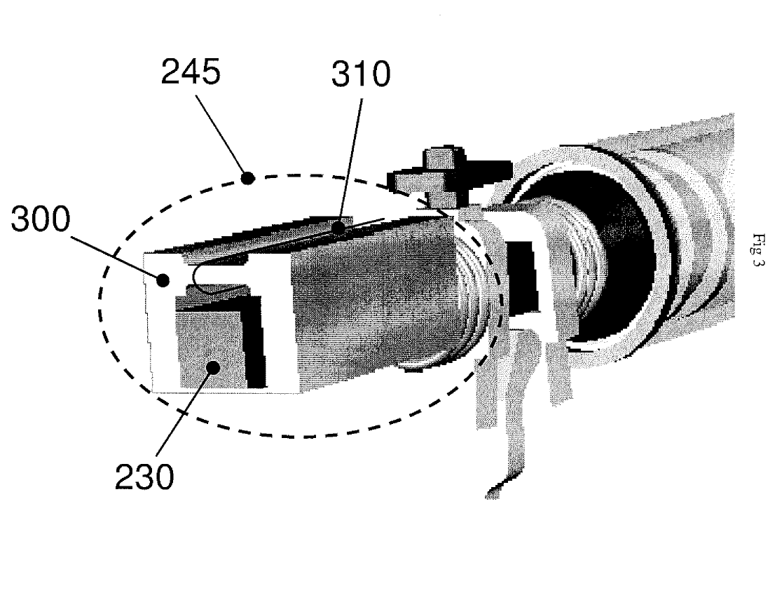

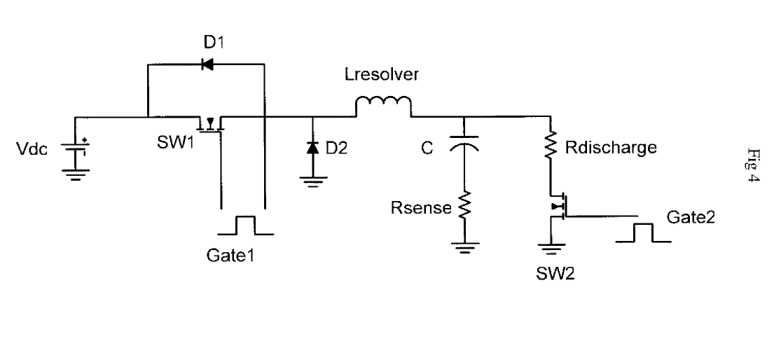

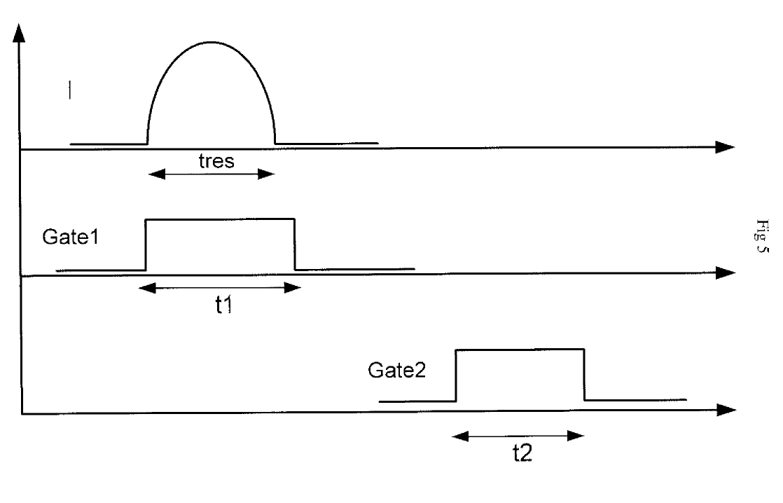

Following is a description of an electromagnetic monitoring mechanism. Linear resolver 245 is positioned around the rear end of the plunger stem 230. Resolver 245, which is illustrated in detail in FIG. 3, is comprised of windings 310 and a core 300 which houses the rear end of plunger stem 230. Linear resolver 245 measures the levels of electromagnetic inductance inside it. For this purpose plunger stem 230 is made from a ferromagnetic material. At its initial position the plunger stem is inline with the back end of linear resolver 245. When the device is in operation and plunger 230 is pushed towards the syringe the overall length of stem 230 inside core 300 changes accordingly. This causes a proportional change in the level of the electromagnetic inductance measured by linear resolver 245. Whenever the expected change in the level of the electromagnetic inductance does not occur the user is notified that the pump is not operating properly. FIG. 4 is an illustration of the electric circuit of the linear resolver and FIG. 5 is an illustration of the input waveform and of the output waveforms of the linear resolver at its two gates.

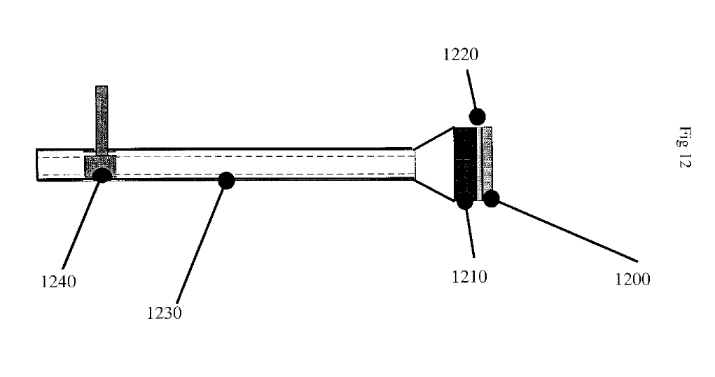

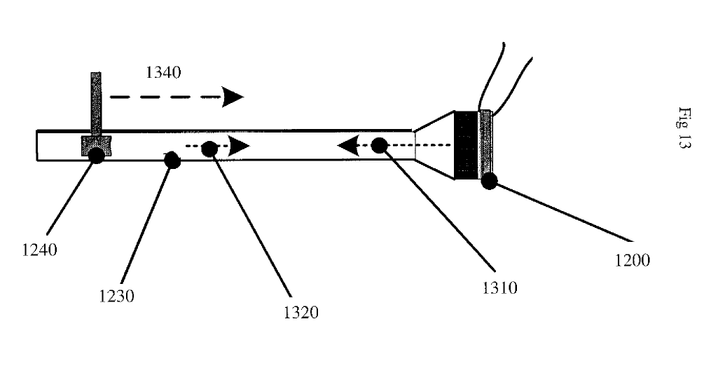

An acoustic resolver is illustrated in FIGS. 12 to 15. As illustrated in FIG. 12 it consists of a piezoelectric transducer 1220, an acoustical wave guide 1230, an acoustical mirror 1240, a first matching layer 1210 and a second matching layer 1200. FIG. 13 illustrates the acoustic resolver in operation. The piezoelectric transducer 1220 transmits a short acoustical pulse of about 100 nsec toward mirror 1240. Transmitted pulse 1320 advances toward mirror 1240 within the medium inside wave guide 1230. The medium inside wave guide 1230 can be air, other types of gas, and different kinds of liquids or solid materials. When pulse 1310 meets mirror 1240, most of its energy is reflected back due to mismatch between the acoustical impedances of mirror 1240 and the medium inside wave guide 1230. As a consequence of the reflection, the pulse travels back 1320 toward piezoelectric transducer 1220. Piezoelectric transducer 1220 behaves both as a transmitting element and receiving element. When acoustical pulse 1320 hits piezoelectric transducer 1220 piezoelectric transducer 1220 vibrates and generates an electrical signal. By measuring the time interval between the moment the pulse was transmitted from piezoelectric transducer 1220 to the moment it hit it again, and by knowing the sound velocity through the medium, it is possible to calculate distance 1340 of mirror 1240 from piezoelectric transducer 1220. Since mirror 1240 is attached to the plunger 230, distance 1340 gives an accurate indication as for the progress of plunger 230. The wavelength of the acoustic signal influences the detection resolution and it should be small comparison to the minimum detectable length. It is preferred to include matching layer 1210 in front of piezoelectric element 1220 in order to match the acoustical impedances of piezoelectric transducer 1220 to that of the medium inside wave guide 1230. Second matching layer 1200 is a backing layer which shortens the vibration tail when the electric pulse is stopped. Wave guide 1230 may be omitted but it increases the signal level detected by piezoelectric transducer 1220. The shape of piezoelectric transducer 1220 can be a rectangular, a disc or the like.

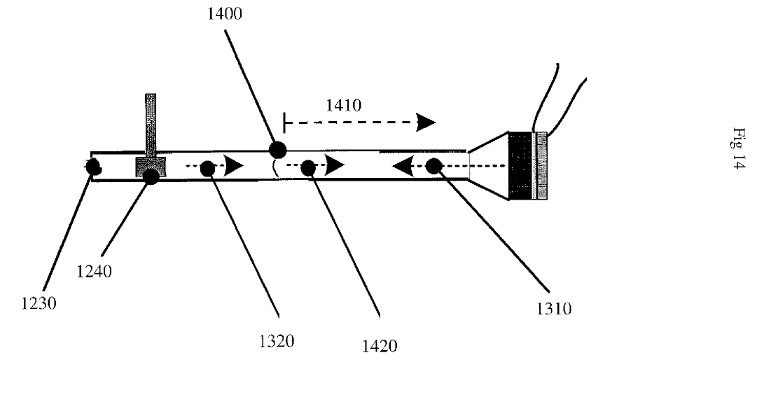

Since the acoustic wave velocity depends on the temperature, it is preferred to measure the temperature and refer to it during the calculations. Alternatively, as illustrated in FIG. 14, a mechanical obstacle 1400 may be positioned in the wave guide 1230, in the path of the transmitted acoustic pulse 1310 at a fixed known point 1410, so it may generate a reference reflection 1420. The time interval needed for the transmitted pulse 1320 to travel from the transmitter to obstacle 1400 and back to the transmitter 1200 may be used as a reference, which in comparison to it distance 1340 to mirror 1240 is calculated.

Also within the scope of the present invention is a control unit which allows users to program the device and receive information regarding its operation. Additionally, the control unit may communicate with a glucose sensing mechanism to form a close loop system, in which the pump delivery rate is dependent on the information coming from the glucose sensor. The control unit may be an integral part of the device, but it may also be a separate unit which communicates with the device via wireless communication. In the latter case the control unit can be carried on the patient's body in an easy to access place, such as on the wrist or in the pocket.

The significantly reduced size and weight of the device allow it to be attached directly to the body of the patient as a small patch, very close to the point of entry of the tube into the body. Shortening the length of the flexible tube leading the medication from the device to the body of the patient to a couple of centimeters reduces the probability of tube-related malfunctions. Such malfunctions may include blockages caused by folds, clutters and medication residues in the tube. In addition, a much smaller amount of medication is needed to perform the prime procedure, and it eliminates the probability of accidental pulling of the tube by patient and others. Attaching the device directly to the body of the patient makes the device much easier to use and to conceal, and does not impose limitations on the movement and mobility of the user.

Flexible tube 160 which leads the medication from the device to the patient may connect to the patient using a standard infusion needle, a transdermal disposable passive patch or an active transdermal sonophoretic patch. The passive patch consists of an array of a large number of very short needles, typically less than 100 micrometers in length. The patch is then located on the skin and the needles particles penetrate the outer layers of the skin, enabling the medication delivery through the skin barrier into the tissue. The needles may be designed to lead the medication through them to the tissue, similarly to standard needles, or, alternatively, the needles may be designed to lead the medication to the tissue on their outer envelope. The needle array may be designed to mechanically vibrate thus enhancing the delivery of medication along the needles to the tissue. The vibration may be generated by a piezoelectric element located on the array patch, by electromagnetic vibrating element or by any other means. For ensuring the safe operation of the device, the patch may communicate with the pump, and alert when unexpected instances occur, such as when the pad is disconnected from the skin.

An active transdermal sonophoretic patch for delivering the medication increases the permeability of the skin by transmitting ultrasound waves, such as transmitting waves in the range of 20 khz-100 khz to the skin. The patch consists of an array of piezoelectric or ferroelectric elements, like piezoelectric or PVDF, which are driven by a power amplifier. The power amplifier generates interleaved electrical signals to the transmitting elements, so current drawn by the power amplifier from the battery contains less ripple and harmonics. The medication enters the patient body tissue through pores which are generated or enlarged by the acoustic radiation being transmitted by the transmitting elements. The active patch may communicate with the pump in order to synchronize its working profile.

While the above description contains many specifications, these should not be construed as limitations on the scope of the invention, but rather as exemplifications of the preferred embodiments. Those skilled in the art will envision other possible variations that are within its scope. Accordingly, the scope of the invention should be determined not by the embodiment illustrated, but by the appended claims and their legal equivalents.

What is claimed is:

1. A micro pump device for dispensing proportioned quantities of medical fluid by applying pulsed pressure on the plunger stem of a syringe containing medical fluid which is injected through a syringe-tube connector to the body of the patient, said device comprised of:
a levering mechanism for applying pressure on the plunger stem in the direction of the tube of the syringe, wherein said lever mechanism includes a lever revolving around a fixed axis and two unidirectional stoppers, a front stopper and a rear stopper, wherein the lever movement applies pressure on said front stopper;
an expending actuator means for applying pressure on the levering mechanism, wherein the activation of the actuator is controlled by a programmable logic module.

2. The micro pump device of claim 1 wherein the first end of said lever is in contact with the actuator and the second end is in contact with said front unidirectional stopper.

3. The micro pump device of claim 1 wherein the unidirectional stoppers comprise a loop placed around the plunger stem having an inner diameter slightly larger then the outer diameter of the plunger stem, and a spring, wherein the angle of loops in relation to plunger stem determine the direction of the stopper.

4. The micro pump device of claim 3 wherein the spring of front unidirectional stopper returns said loop to its initial position in relation to said lever.

5. The micro pump device of claim 1 wherein said rear unidirectional stopper prevents a backwards motion of the plunger.

6. The micro pump device of claim 1 further comprising a knob for releasing the hold of said second unidirectional stopper on the plunger stem by slightly changing the angle of the loop of the rear stopper and releasing its grip on said plunger stem, enabling the refill of said syringe tube or the replace of it, wherein upon releasing said knob the spring of the rear stopper returns the loop of the rear stopper back into place in relation to plunger stem.

7. The micro pump device of claim 1 further comprising a sensor for measuring the force of the actuator, said sensor is positioned between the plunger stem and the seal of the syringe.

8. The micro pump device of claim 7 wherein said sensor is a piezoelectric transducer.

9. The micro pump device of claim 1 further comprising a sensor for monitoring the movement of said plunger stem, enabling a continuous calculation of the pump flow rate.

10. The micro pump device of claim 9 wherein said sensor is an optical sensor.

11. The micro pump device of claim 9 wherein said sensor is an electromagnetic sensor positioned around the rear end of the plunger stem.

12. The micro pump device of claim 11 wherein the electromagnetic sensor is a resolver comprised of: windings and a core.

13. The micro pump device of claim 1 wherein said device is connected to a patch for delivering said medication into the body of the user.

14. The micro pump device of claim 13 wherein said patch is an active transdermal multi needle array patch.

15. The micro pump device of claim 14 wherein said multi array patch vibrates mechanically to improve the medication delivery of the patch.

16. The micro pump device of claim 15 wherein said multi array patch contains a piezoelectric vibrating element or an electromagnetic vibrating element.

17. The micro pump device of claim 13 wherein said delivery patch communicates with said pump device and alerts in cases of unexpected events.

18. The micro pump device of claim 1 wherein said axis is a unidirectional rotation bearing.

19. The micro pump device of claim 1 further includes a soft sealing around the plunger stem that prevents contamination from entering the mechanism from said syringe tube.

20. The micro pump of claim 1 further including a piezoelectric sensor, an electric circuit and a module for performing static measurement of the pressure, wherein said electric circuit and module electrically stimulate for a defined period the piezoelectric sensor with an appropriate signal which charges the RLC of the first motional branch of the sensor, at the end of the stimulation a zero voltage is forced on the electrical electrodes, causing the pre-charged RLC to discharge through the short circuit in an alternating current whose envelope decays exponentially, wherein the level of the exponential decay constant level of the exponential decay represents the level of mechanical stress exerted on said element.
